(19) United States
(12) Patent Application Publication
(10) Pub. No.: US 2021/0290421 A1
(43) Pub. Date: Sep. 23, 2021

(54) POSTURE CORRECTING APPARATUS

(71) Applicant: Timothy Ward Clarke, Mechanicville, NY (US)

(72) Inventor: Timothy Ward Clarke, Mechanicville, NY (US)

(21) Appl. No.: 17/209,526

(22) Filed: Mar. 23, 2021

Related U.S. Application Data
(60) Provisional application No. 62/993,555, filed on Mar. 23, 2020.

(51) Int. Cl. A61F 5/02

(52) U.S. Cl. CPC A61F 5/026

(57) ABSTRACT

An apparatus for enabling correct posture of a user is shown and described. The apparatus features a first wearable harness, which, when worn by a user is proximate to the user's hip. The apparatus also has a second wearable harness, which, when worn by a user is proximate to the user's shoulder blades. A strap element connects the first wearable harness to the second wearable harness. Additionally, the apparatus has a length adjusting element affixed to either the first wearable harness and or the second wearable harness. The length adjusting element permits the user to adjust a length of the strap element between the first and second wearable harnesses.

### CROSS-REFERENCE TO RELATED APPLICATIONS

This application claims the benefit of U.S. provisional application 62/993,555, filed Mar. 23, 2020 and entitled, “POSTURE CORRECTING APPARATUS”, the likes of which is hereby incorporated by reference herein in its entirety.

### STATEMENT REGARDING FEDERALLY SPONSORED RESEARCH OR DEVELOPMENT

Not applicable.

### INCORPORATION BY REFERENCE OF MATERIAL SUBMITTED ON A COMPACT DISC

Not applicable.

### TECHNICAL FIELD

The present invention relates to exercise equipment and posture support, and more specifically, to the field of harnesses worn on the body configured to correct posture and alignment when enduring physical activity.

### BACKGROUND

Lower back strain and other back related injuries are considered some of the most common injuries associated with physical fitness, especially physical fitness involving weights. With fitness becoming more popular than ever, there has been an increase in the amount of back related injuries caused by physical fitness involving weights. In particular, individuals who utilize free weights are commonly exposed to lower back strain and other back related injuries when performing popular exercises such as deadlifts, seated rows, bent over rows, and others. A few typical causes for lower back strain and other back related injuries endured during these exercises are the lifter rounding their back and bending over too far at the hips before lifting the weight, the lifter holding the weight too far from their body, or the lifter hyperextending their lower back. All of these causes can be attributed to the lifter having an incorrect posture when performing these exercises.

One method of circumventing this issue is to simply apply the correct posture when performing these exercises. However, during a lifting session, the lifter may start with the correct posture to avoid injury, but the correct posture may fade once the lifter becomes fatigued from multiple repetitions or sets; thus, increasing the likelihood of sustaining back injury. Another approach is for the lifter to wear a back brace during exercise that is configured for lumbar support. However, a common critique of back braces is that not only are they difficult for an individual of average or less than average flexibility to use, but also they are incredibly uncomfortable to wear during exercising due to failure to meet within the middle of a compressed fit configured to align the spine mid-exercise and a loosened comfortable fit when the lifter is resting between sets. Furthermore, these back braces are not configured to switch between an engaged and disengaged functionality. For example, if a lifter desires a disengaged functionality for the back brace, then the lifter must either detach components of the back brace or take off the back brace altogether. As a result, there exists a need for improvements over the prior art and, more particularly, for a better apparatus to correct the posture of a lifter and align with the spine when the lifter is participating in exercises that are commonly associated with a back strain and other back injuries.

### BRIEF DESCRIPTION OF THE DRAWINGS

The accompanying drawings, which are incorporated in and constitute part of this specification, illustrate embodiments of the invention and together with the description, serve to explain the principles of the disclosed embodiments. The embodiments illustrated herein are presently preferred, it being understood, however, that the invention is not limited to the precise arrangements and instrumentalities shown, wherein:

FIG. 1a is a rear-perspective view of an apparatus for correcting the posture of a user being worn by a user, according to an example embodiment;

FIG. 1b is front-perspective view of an apparatus for correcting the posture of a user being worn by a user, according to an example embodiment;

FIG. 1c is a zoomed-in cutaway view (cutaway to illustrate the wires) of a tightening device of an apparatus for correcting posture, according to an example embodiment;

FIG. 1d is a zoomed-in cutaway view (cutaway to illustrate the wires) of a strap element attached to a first wearable harness and second wearable harness, the strap element having wires that attach to a tightening device, according to an example embodiment;

FIG. 2a is a front-perspective cutaway view (with the front housing and handle/spindle shown in FIG. 2c removed) illustrating internal components of a tightening device, according to an example embodiment;

FIG. 2b is a rear-perspective view of a rotating gear attached to a wire for a tightening device, according to an example embodiment;

FIG. 2c is a perspective side exploded view of a tightening device, according to an example embodiment;

FIG. 3a is a top perspective view of the apparatus for correcting the posture of a user, according to an example embodiment of the present invention;

FIG. 3b is a zoomed-in or enlarged perspective view of a length adjusting element of the apparatus for correcting the posture of a user, according to an example embodiment of the present invention;

FIG. 3c is a perspective view of the internal components of the tightening device shown in FIG. 3d;

FIG. 3d is a front-perspective view of a tightening device attached to a strap element;

FIG. 4 is a front perspective view of the apparatus for correcting the posture of a user, with a zoomed in section of the area within perimeter AA, according to an example embodiment of the present invention;

FIG. 5 is a top perspective view of a ratcheting system associated with a length adjusting element of an apparatus for correcting the posture of a user, according to an example embodiment; and,

FIG. 6 is a side perspective view of the apparatus for correcting the posture of a user in a locked position worn by the human wearer, according to an example embodiment of the present invention.

### DETAILED DESCRIPTION

The following detailed description refers to the accompanying drawings. Whenever possible, the same reference numbers are used in the drawings and the following description to refer to the same or similar elements. While disclosed embodiments may be described, modifications, adaptations, and other implementations are possible. For example, substitutions, additions, or modifications may be made to the elements illustrated in the drawings, and the methods described herein may be modified by substituting reordering or adding additional stages or components to the disclosed methods and devices. Accordingly, the following detailed description does not limit the disclosed embodiments. Instead, the proper scope of the disclosed embodiments is defined by the appended claims.

The disclosed embodiments improve upon the problems with the prior art by providing an apparatus for correcting the posture of a user when engaged in exercising so that the user does not sustain back strain or any other applicable injury.

The present invention improves over the prior art by providing a first wearable harness configured to be affixed to the hip section of the user, a second wearable harness configured to be affixed to the upper body section of the user, a strap element configured to be integrated with the first and second wearable harnesses, and at least one length adjusting element configured to be coupled to the strap element and associated with at least one of the wearable harnesses.

The present invention also improves over the prior art by providing a first wearable harness, wherein, when worn by the user, the first wearable harness is proximate to a hip section of the user, a second wearable harness, wherein, when worn by the user, the second wearable harness is proximate to shoulder blades of the user, a strap element connecting the first wearable harness to the second wearable harness, and at least one length adjusting element, wherein the at least one length adjusting element adjusts a length of the strap element defined by a distance between the first wearable harness and the second wearable harness, and wherein the at least one length adjusting element is affixed to at least one of the first wearable harness and the second wearable harness.

Additionally, the disclosed embodiments improve over the prior art by providing a locked and unlocked position, wherein in the locked position the strap element spans the back of the user and provides a tension between the first and second wearable harnesses forcing the user to maintain a correct alignment of the user's spine when performing lifting exercises that require bending at the knees of the user, and in the unlocked position the length adjusting element allows the length of the strap element to be changed.

### i. Apparatus and Method of Use

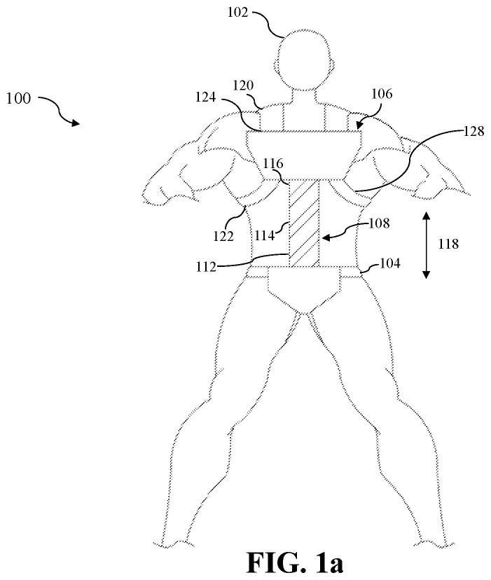

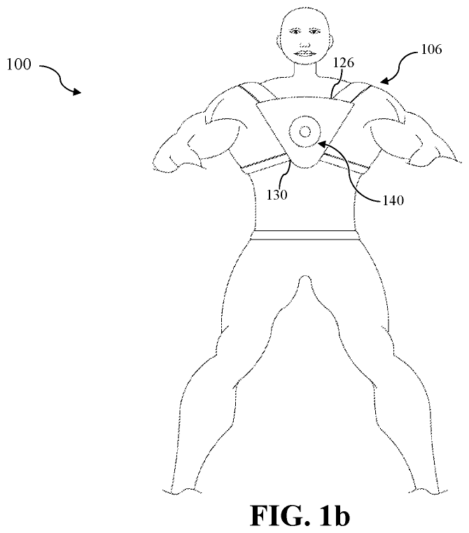

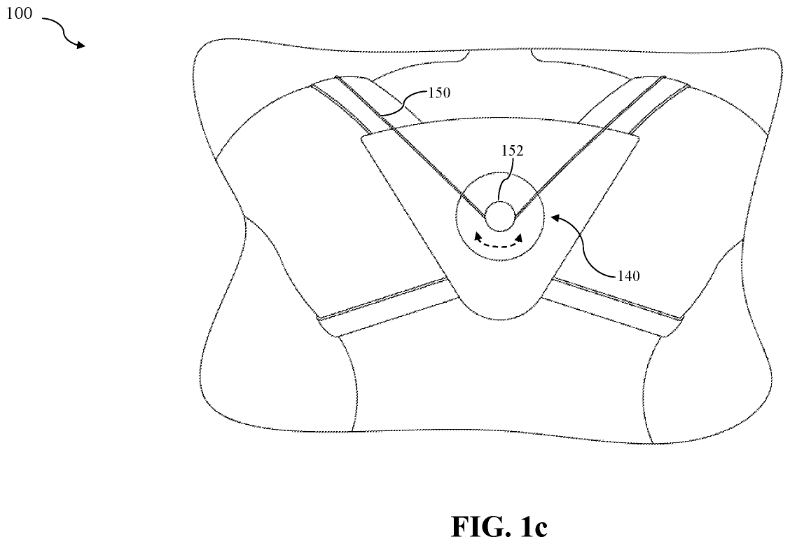

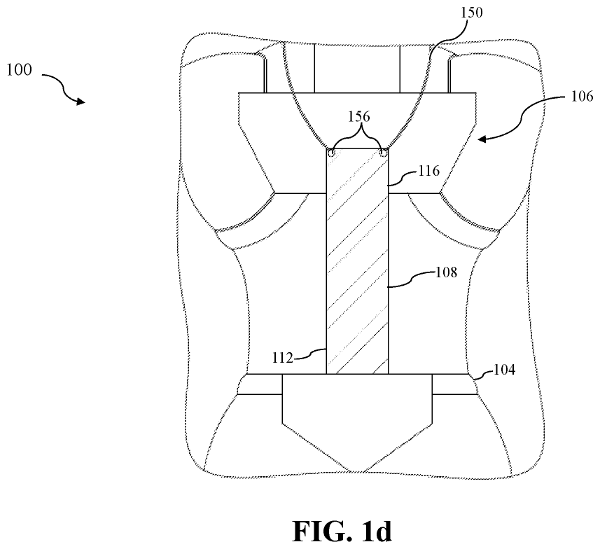

Referring now to the Figures, FIG. 1a is a perspective view of an example embodiment apparatus 100 for correcting the posture of a user 102 and illustrated as being worn being by the user 102. The apparatus 100 comprises a first wearable harness 104 configured to be affixed to the hip section of user 102, a second wearable harness 106 configured to be affixed to the upper body section of user 102, a strap element 108 configured to be integrated with first wearable harness 104 and second wearable harness 106, and a length adjusting element 110 configured to be coupled to the strap element 108 and associated with first wearable harness 104, second wearable harness 106, or both. More specific structural descriptions and characteristics of the components of the apparatus are described below.

When the apparatus 100 is worn by a user, first wearable harness 104 spans the waistline or hip area of user 102, and second wearable harness 106 spans the upper body of user 102. In one embodiment, first wearable harness 104 continuously spans the waist and pelvic area of user 102, and second wearable harness 106 completely spans the shoulders, shoulder blades, and chest area of user 102. As discussed in greater detail below, the first wearable harness 104 and second wearable harness 106 may be secured to the body via at least one strap on each harness 104, 106 configured to attach the first wearable harness 104 and the second wearable harness 106 to the user 102's 100 body.

The strap element 108 connects the first wearable harness 104 and the second wearable harness 106, thereby spanning the back of user 102. As discussed in greater detail below, strap element 108 exerts a tension force 118 that aligns the spine of the user in a proper posture. Strap element 108 may be disposed (e.g., permanently affixed) anywhere on first wearable harness 104 and/or second wearable harness 106 that allows strap element 108 to span the back of user 102. Alternatively, strap element 108 may be detachable from apparatus 100, allowing user 102 to put on first wearable harness 104 and second wearable harness 106 respectively, then affix strap element 108 to a portion of first wearable harness 104 and a portion of second wearable harness 106. The strap element 108 may be affixed via any applicable mechanism resulting in strap element 108 integrating with first wearable harness 104 and second wearable harness 106 while simultaneously spanning the back of user 102. For instance, a hook and loop configuration may be used. Another method for securing strap element 108 to the first wearable harness 104 may include directly sewing the strap element 108 to the first wearable harness 104.

In one embodiment, apparatus 100 may be assembled entirely in an attached configuration allowing user 102 to simply insert their respective limbs into first wearable harness 104 and second wearable harness 106 (in no particular order), resulting in pre-attached strap element 108 to come into contact with the back of user 102, and subsequently adjusting apparatus 100 via length adjusting element 110 until apparatus 100 is comfortably snug on user 102. In one embodiment, apparatus 100 may be completely disassembled in which first wearable harness 104 and second wearable harness 106 are worn piece by piece in no particular order and strap element 108, which is detached, is affixed to a portion of first wearable harness 104 and a portion of second wearable harness 106 allowing strap element 108 to come into contact with the back of user 102.

In one embodiment, first wearable harness 104, second wearable harness 106, and strap element 108 may be composed of any fabric or any other thin, flexible material that will readily endure stretching and not readily tear when enduring forces and motions inherent to exercising and other applicable physical activities. First wearable harness 104 and second wearable harness 106 may further include padding, cushioning, or any other applicable insulation configured to support a comfortable fit when worn by user 102.

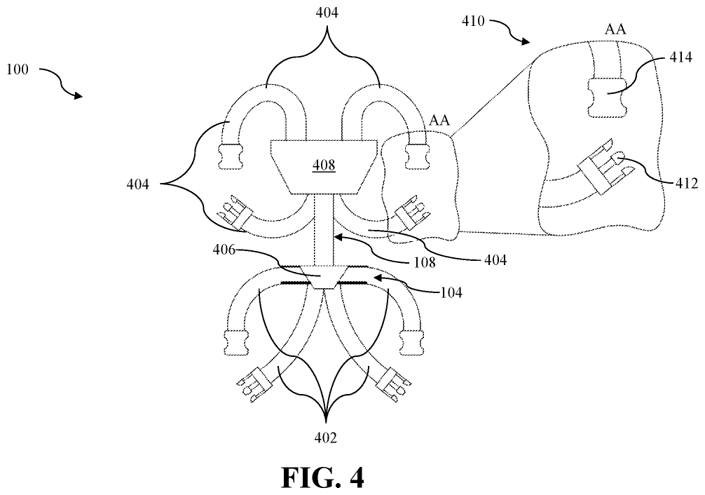

In one embodiment, first wearable harness 104 and second wearable harness 106 are comprise a plurality of panels, which may be transverse, configured to interlock with each other via a hook and loop configuration, such as VELCRO®, or any other suitable mechanism for affixing known in the art. The plurality of panels referred to in greater detail in FIG. 4, are configured to allow the wearing of apparatus 100 on individuals with average or less than average flexibility while simultaneously maintaining a comfortable and secure fitting that allows for a full range of movements without user 102 enduring chaffing or any other applicable issues commonly associated with harnesses.

FIG. 1a depicts user 102 wearing apparatus 100 in a locked position. As described herein, a locked position refers to when middle segment 114 spans the back of user 102, providing a tension force 118 between first wearable harness 104 and second wearable harness 106 that is retained and secured by length adjusting element 110. Conversely, an unlocked position refers to any position in which length adjusting element 110 allows the length of strap element 108 to be extended or shortened. For instance, when in an unlocked position, the user may adjust the length of strap element 108 based on their preference. As described, in the locked position the length of the strap element 108 is fixed, and in the unlocked position the length of the strap element 108 adjusts according to the distance between the first wearable harness 104 and the second wearable harness 106. In this regard, the length of the strap element 108 between the first wearable harness 104 and the second wearable harness 106 is adjustable to properly fit the user 102. As discussed in greater detail below, the apparatus 100 may be configured to switch between the locked position and the unlocked position by the user 102 interacting with at least one of a dial, lever, button, or actuator. Regardless, when in an unlocked position, the strap element 108 is configured to move freely when worn by the user 102.

For illustrative purposes, user 102 may wear apparatus 100 in the locked position even when user 102 is not engaged in lifting exercises that require bending at the knees; thus, allowing apparatus 100 to maintain posture correcting functionality when user 102 is performing other various activities. For example, as illustrated in FIG. 1a, apparatus 100 is fully functional in the locked position wherein tension force 118 is secured and retained by length adjusting element 110 while user 102 is standing upright. In other words, the apparatus 100 may be used for other purposes other than performing a weightlifting exercise, such as when performing an activity such as running.

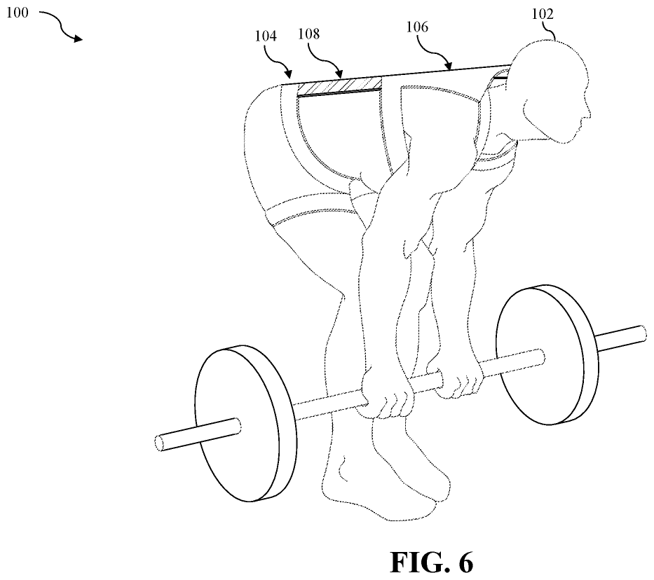

Referring now to FIG. 6, an example embodiment of apparatus 100 in the locked position worn by user 102 performing a bent over barbell row is depicted. In the depicted application, first wearable harness 104 is affixed to the hip section of user 102, and second wearable harness 106 is affixed to the upper body of user 102, allowing strap element 108, in particular middle segment 114, to span the back of user 102. Due to apparatus 100 being in the locked position, strap element 108 is able to provide tension force 118 between first wearable harness 104 and second wearable harness 106; thus, forcing user 102 to maintain correct alignment of the spine of user 102. The combination of strap element 108 aligning with the spine along with the application of tension force 118 prevents user 102 from rounding the back, hyperextending the back, or bending over too far at the hips when user 102 is performing lifting exercises that require bending at the knees of the user.

The method of using an apparatus 100 for correcting posture while performing an exercise may comprise steps of mounting a first wearable harness 104 on a hip section of the user 102, mounting a second wearable harness 106 on an upper body of the user 102, positioning the strap element 108 longitudinally along a back portion of the user 102. The strap element 108 may be positioned in such a manner that the strap element 108 connects the first wearable harness 104 to the second wearable harness 106. As noted above, the strap element 108 defines a length defined by a distance between the first wearable harness 104 and the second wearable harness 106, thus the strap element length is critical to maintaining the correct posture while performing the exercise. In this way, the method may include adjusting the length between the first wearable harness 104 and the second wearable harness 106 so that the strap element 108 forces the user 102 to maintain the correct posture while performing the exercise.

### i. Apparatus Components

As noted above, an apparatus 100 for enabling correct posture of a user 102 generally comprises a first wearable harness 104, a second wearable harness 106, and a strap element 108 connecting the first wearable harness 104 to the second wearable harness 106 and at least one length adjusting element 110. When worn by the user 102, the first wearable harness 104 is proximate to a hip section of the user 102, and the second wearable harness 106 is proximate to shoulder blades of the user 102. The at least one length adjusting element 110 adjusts a length of the strap element 108 defined by a distance between the first wearable harness 104 and the second wearable harness 106. Furthermore, the at least one length adjusting element 110 is affixed to at least one of the first wearable harness 104 and the second wearable harness 106.

The second wearable harness 106 may comprise a first set of straps 120 and a second set of straps 122. The straps (120, 122) enable the user 102 to insert their arms to secure the apparatus 100 to their upper body. When worn by the user, the first set of straps 120 extend from a first straps first end 124 proximate the shoulder blades of the user 102, over trapezius muscles of the user 102, and to a first straps second end 126 proximate a chest of the user 102. Furthermore, when worn by the user the second set of straps 122 extend from a second straps first end 128 proximate the shoulder blades of the user 102, over a ribcage of the user 102, and to a second straps second end 130 proximate the chest of the user 102.

As noted above, the apparatus comprises at least one strap element 108 and the strap element 108, the strap element 108 connects the first wearable harness 104 to the second wearable harness 106. The length adjusting element 110 enables strap element 108 to be incrementally tightened, increasing tension force 118 between first segment end 112 and second segment end 116. When the apparatus is in a locked position/configuration, the apparatus 100, and more specifically the strap element 108, creates a tension force 118 to influence user 102 to maintain a correct alignment of the user's spine. The tension force 118 that aligns the user's spine may be particularly useful when performing lifting exercises that require bending at the knees, such as deadlifts or bent over rows, to name a few.

Referring now to FIG. 4, apparatus 100 is depicted including first wearable harness 104 and second wearable harness 106 comprising a plurality of first wearable harness panel straps 402, a plurality of second wearable harness panel straps 404, a belt or waist panel 406, and an upper body panel 408. In one embodiment, each of the plurality of harness panels straps are configured to attach the first and second wearable harnesses to the body of user 102. In one embodiment, the plurality of first wearable harness panel straps 402 and the plurality of second wearable harness panel straps 404 comprise a fastener mechanism 410, including a male end 412 and a female end 414 configured to interlock with each other in order to secure placement of the panels on user 102. In one embodiment, fastener mechanism 410 may be a hook and loop configuration, such as VELCRO®, or any other suitable mechanism for affixing known in the art that allows user 102 to secure apparatus 100 to the body. In one embodiment, any of the wearable harness panels may be detachable from belt or waist panel 406 and upper body panel 408. In one embodiment, belt or waist panel 406 is positioned on the rear waist area of user 102 and upper body panel 408 is positioned on the upper back area of user 102 wherein the plurality of first wearable harness panel straps 402 may traverse the thighs of user 102 starting at the hamstring area and the plurality of second wearable harness panel straps 404 may traverse over and/or under the shoulders and arms of user 102. User 102 may decide the optimal fitting of the wearable harness panels around extremities and secure the fitting by interlocking male end 412 and female end 414 of fastener mechanism 410. In one embodiment, the length of any of the wearable harness panel straps may be adjustable in order to support proper fitting among various body types.

### iii. Length Adjusting Elements

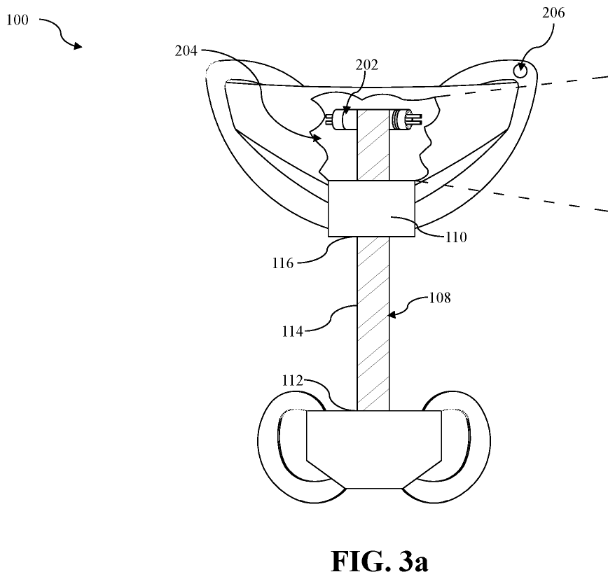

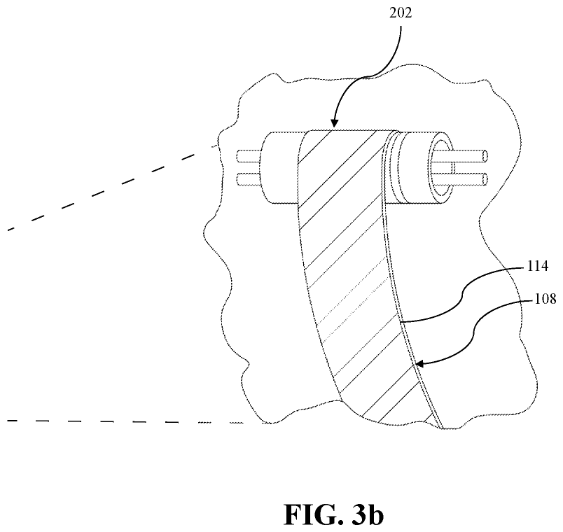

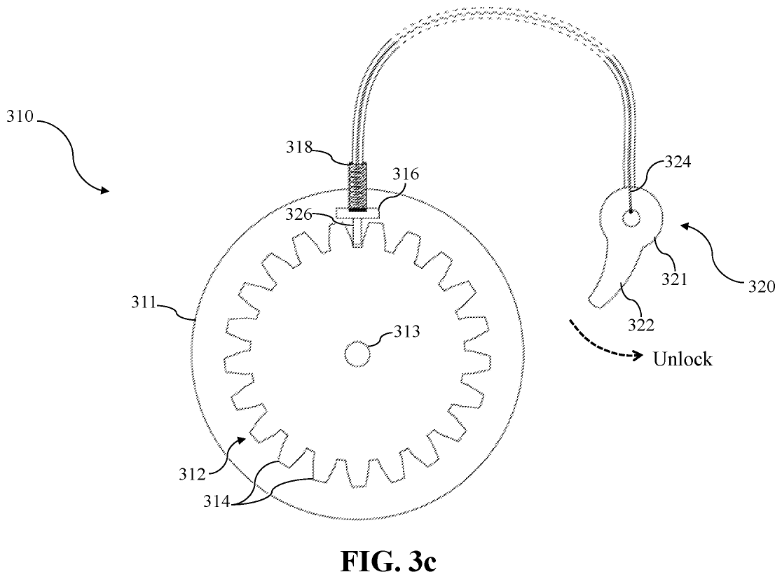

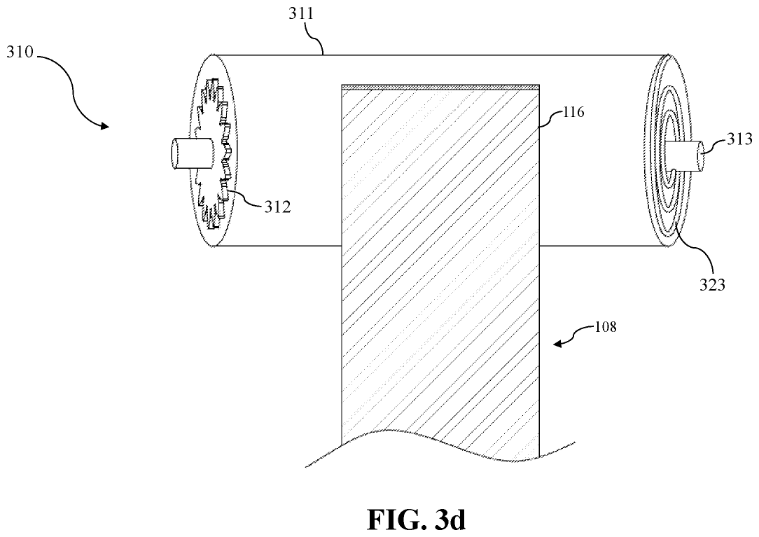

As noted above, the apparatus 100 for correcting posture generally comprises a length adjusting element. As will become apparent by the foregoing description, there are numerous possibilities for a suitable length adjusting element. For instance, the length adjusting element may be located on a front of the apparatus (see FIG. 1b), or on the back of the apparatus (see, e.g., FIG. 3b). In this regard, the length adjusting element is generally capable of adjusting the fit of the apparatus on the user. The fit of the apparatus on the user can be determined, for instance, by the length of strap element 108, which as described above, creates a tension force 118 that aligns the user's spine.

With reference to FIGS. 1b-1c, in one embodiment, the length adjusting element is mounted on the second wearable harness 106 and comprises a tightening device 140. As illustrated, when worn by the user 102, the tightening device 140 is proximate the chest of the user 102. In one embodiment, the tightening device 140 comprises at least one wire 150. The at least one wire 150 may be embedded in each of the first set of straps 120 of the second wearable harness 106. The at least one wire 150 may be engaged with a rotating spindle 152 that when rotated in a first direction 214 tightens the at least one wire 150, thereby shortening the length of the strap element 108. As illustrated in FIGS. 1b and 1c, the tightening device 140 that is considered as being a part of the length adjusting element 110 (or may be considered as being the length adjusting element 110 itself) is located on a front of the second wearable harness 106. As shown in the cutaway view of FIG. 1c, the at least one wire 150 is visible to illustrate the design. As one may appreciate, the at least one wire 150 may be obfuscated by being embedded in the first set of straps 120 of the second wearable harness 106. The at least one wire 150 may also be embedded by, for instance, covering the at least one wire 150 with a sheath that traverses the length of the at least one wire 150.

The at least one wire 150 may traverse (e.g., through the first straps 120) of the second wearable harness 106, over the trapezius muscles of the user, and down toward the shoulder blades of the user. Near the shoulder blades of the user, the at least one wire 150 may terminate at a second end segment 116 of strap element 108. In this regard, strap element 108 may include at least one hole 156 for receiving the end of the at least one wire 150, thereby securing the at least one wire 150 to the strap element 108 to adjust the strap element's 108 length. However, other means for attaching the wire 150 to the strap element, such as fasteners (including bolts, screws, magnets and other types of items), may be used and are within the spirit and scope of the present invention.

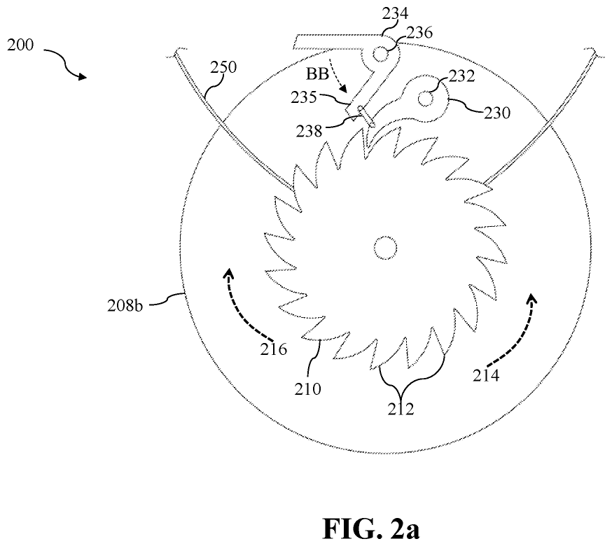

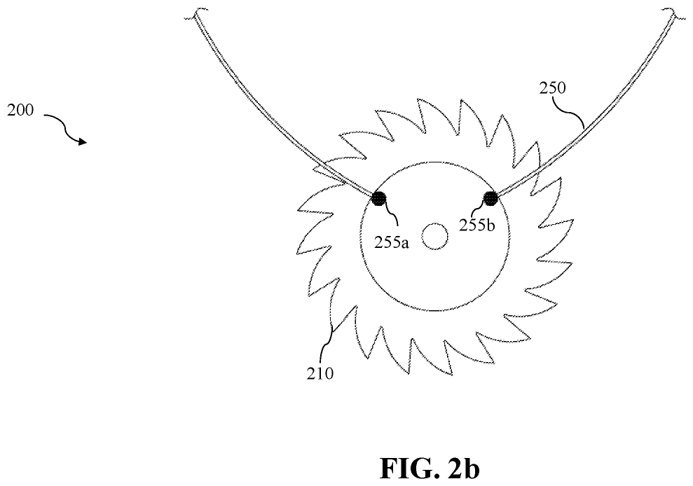

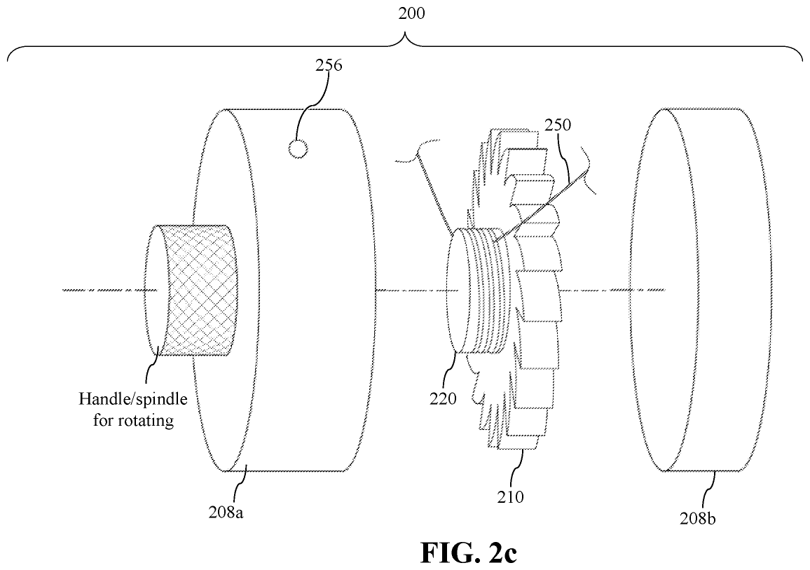

Regarding the internals of the tightening device 140 illustrated in FIGS. 1b-1c, FIGS. 2a-2c show an embodiment of the internal components of a tightening device 200. As shown in FIG. 2a, the tightening device 200 embodiment comprises a rotating gear 210, a drum 220, a pawl 230, a lever 234, and at least one wire 250. As shown in the illustrated embodiments, two wires 250 (i.e., one for the left side and one for the right side) are shown. The wires 250 terminate inside the tightening device via holes 255a, 255b inside the drum 220. Additional holes 256 in the front housing 208a enable the wires to engage the drum 220 and rotating gear 210. Lastly, the user may adjust the tightness using spindle 207 on the outer portion of the tightening device 140.

As shown by the front-view of tightening device 200 shown in FIG. 2a, rotating gear 210 comprises a plurality of teeth 212, the plurality of teeth 212 as illustrated are shown as biasing in one direction, such that pawl 230 can smoothly rotate in a single direction (first direction 214) to increase tightening forces on the at least one wire 250. As the rotating gear rotates in the tightening direction 214, the at least one wire 150 wraps on to the drum 220, which is attached to the rotating gear 210. When the user has tightened the apparatus to their preferences, the rotating gear 210 is prevented from rotating in the loosening direction (second direction 216) by the engaged pawl 230.

The pawl 230 engages the plurality of teeth 212 due to a first force provided by at least a connector 238. Connector 238 provides a tension force on the pawl 230, thereby biasing the pawl 230 towards engagement. The connector 238 may be any suitable feature, such as a first spring element (e.g., a compressive spring, a leaf spring). In other embodiments, the connector may be an elongated rod that connects a portion of the lever to the paw. Lever 234 may have a portion outside the rear housing 208b and the front housing 208a (omitted from the view in FIG. 2a), and this portion may be grasped by the user and lifted such that the pawl may disengage the rotating gear 210, allowing the rotating gear 210 to rotate in the loosening direction 216. Additional spring elements may be used in such an arrangement. For instance, lever 234 may have an additional spring element, which may be included in the mounting 236 for the lever 234 that is configured to rotate the lever such that a section 235 of the lever rotates inward in the direction of line BB so that the pawl is continuously biased and engages with the teeth, unless a force acts on the lever to move the pawl away from and disengage the teeth.

The mounting 236 may be, for instance, a spindle mounted on or integral with the rear housing 208b, and the spindle may include a spring element wrapping around the spindle and engaged with the lever 234 (e.g., a torsion spring). This type of spring element may additionally provide biasing forces (e.g., in addition to connector 238) to engage the pawl 230. Pawl 230 may include a similar mounting 232 that includes an additional spring element to bias the pawl 230. Lever 234 is connected to the pawl 230, such that the lever 234 disengages the pawl 230 from the plurality of teeth 212 when a second force acts on the lever 234 that is greater than the first force. The second force may be, for instance, the force stored in the tightened wire 250 that readily causes the wire 250 to loosen when the pawl 230 is disengaged. Moreover, the first force may be a sum combination of certain forces, such as forces produced through one or more forces from connector 238, and any additional spring elements found in the mountings 232, 236. Additional forces may contribute, such as forces from any securing devices used to secure the latch 234 in the locked position. For instance, latch 234 may be strapped down to the outer portions of tightening device using VELCRO®, or any other suitable means for securing the latch.

As noted above, at least one wire 250 may be used in the tightening device 200 design. The at least one wire 250 may be embedded in each of the first set of straps 120 of the second wearable harness 106. The at least one wire 250 is attached to the drum 220. For instance, the wire 250 is attached to the drum by wrapping around the drum. More specifically, the wire 250 may wrap around grooves that are present in the drum. As noted above, when the at least one wire 250 wraps on to the drum 220, the drum 220 rotates with the rotating gear 210 in the first direction 214, thereby tightening the at least one wire 250 and shortening the length of the first set of straps 120. Furthermore, when the at least one wire 250 unwraps from the drum 220, the drum 220 rotates with the rotating gear 210 in the second direction 216 thereby loosening the at least one wire 250 and increasing the length of the first set of straps 120.

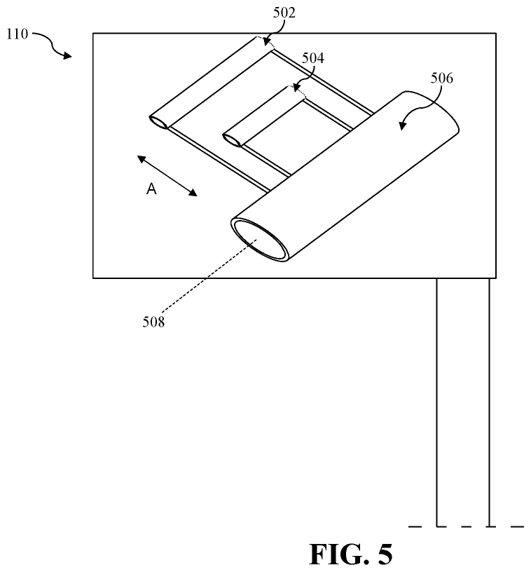

As noted above, numerous types and styles of length adjusting element(s) may be used to affect the length of strap element 108. An embodiment of length adjusting element 110, referred to in greater detail in FIG. 5, may be integrated with strap element 108, which is associated with a ratcheting strap system configured to support extension and shortening of strap element 108 depending upon the apparatus 100 being in the unlocked position. Length adjusting element 110 may include a ratcheting system 204, including a spindle, reel, spool, or any other applicable mechanism configured to provide adjustable functionality to strap element 108. However, it is understood that other ratcheting systems may be used and are within the spirit and scope of the present invention.

For example, the attaching system may include a ratcheting system similar to the ratcheting system used in a ratchet socket wrench. In such an embodiment, a dial allows the ratcheting system to be moved between the shortening position/neutral position or unlocked position and the locked position. When the dial is in the neutral position, the ratcheting system allows movement such that the length of strap between the first wearable harness and second wearable harness to increase or decrease. When the dial is in the neutral or unlocked position, the spindle or spindle of ratcheting system allows the strap to be shortened and lengthened freely. This allows the user or wearer to move freely and for the user to find their correct posture. When the dial is in the locked position, the spindle is fixed and thus the ratcheting system prevents the length of the strap between the first wearable harness and second wearable harness from being shortened or lengthened. In the locked position, the fixed strap provides a tension between the first and second wearable harnesses forcing the user to maintain a correct alignment of the user's spine when performing lifting exercises that require bending at the knees of the user.

Additionally, in another embodiment, the ratcheting system may include a ratcheting system similar to those used in tie down or strap like systems that allow a user to shorten the length of a strap, lock a strap in a certain position and allow the strap to spool freely by use of a lever. In such embodiments, the lever may be moved between a locked position, an unlocked position and a shortening position. When the lever is in the shortening position, the ratcheting system allows the length of the strap to be shortened, but not increased. When the lever is in the unlocked position, the ratcheting system allows the spool on which the strap is spooled to freely spool so that length of the strap to be increased. When the lever is in the locked position, the ratcheting system prevents either lengthening or shortening of the strap.

Length adjusting element 110 may include a buckle or other applicable mechanism for strap element 108 to pass through configured to lock and secure strap element 108 at the length preferred by user 102. In one embodiment, strap element 108 further includes a first end segment 112 configured to be attached to first wearable harness 104, a middle segment 114 configured to span the back of user 102, and a second end segment 116 configured to be attached to second wearable harness 106.

Referring now to FIG. 3a, a top perspective view of apparatus 100 is depicted, including length adjusting element 110 comprising a spindle 202, ratcheting system 204, and a dial 206 configured to switch apparatus 100 between the locked position and the unlocked position. In one embodiment, dial 206 may be a lever, button, actuator, or any other mechanism configured to control activation of length adjusting element 110 so that strap element 108 may switch between the locked position and the unlocked position via interaction between user 102 and the applicable mechanism. In one embodiment, length adjusting element 110, spindle 202, ratcheting system 204, and dial 206 functional as an integrated unit. In application, user 102 wears apparatus 100 as described above wherein apparatus 100 is in the unlocked position indicated by dial 206 in the applicable setting and strap element 108 is already partially fed into an opening of ratcheting system 204 enabling a pulley like configuration with spindle 202 that supports slack in strap element 108. Whenever there is slack associated with strap element 108, it is an indicator that apparatus 100 is the unlocked position allowing the length of strap element 108 to be freely changed by user 102 via length adjusting element 110. At this point, user 102 adjusts the length of strap element 108 by incrementally tightening and loosening strap element 108 via a pushing or pulling motion exerted on strap element 108 resulting in strap element 108 being adjusted through spindle 202 until a snug yet comfortable fitting of apparatus 100 according to the preferences of user 102 is obtained. During this adjustment process, retaining forces associated with ratcheting system 204 exceed tension force 118 because the buckle associated with ratcheting system 204 has not been engaged. Once the desired fitting of apparatus 100 is achieved, user 102 interacts with dial 206 and switches apparatus 100 from the unlocked position to the locked position resulting in the buckle being engaged, ratcheting system 204 winding excess portions of strap element 108 onto spindle 202 until tension force 118 exceeds the retaining forces associated with ratcheting system 204; thus, eliminating slack associated with strap element 108. Therefore, tension force 118 keeps middle segment 114 securely in contact with the back of user 102, resulting in middle segment 114 spanning the back and correcting the alignment of the spine while being disposed between first and second wearable harnesses.

Referring now to FIG. 3b, dial 206 is engaged in the locked position while spindle 202 and ratcheting strap system 204 maintains the current length of strap element 108 and prevents the length of strap element 108, in particular middle segment 114, from being altered. In other words, it is imperative that the length of middle segment 114 remains unchanged in order to not alter the positions of the first end segment 112 attached to first wearable harness 104 and the second end segment 116 attached to second wearable harness 106. This allows retention of the position and spanning of middle segment 114 along the back even when user 102 is performing a full range of motions.

With reference now to FIG. 3c-3d, an embodiment for a length adjusting element 310 is shown. As illustrated, the length adjusting element 310 comprises a gear 312 having a plurality of teeth 314, a mounting structure 316, a spring 318 attached to the mounting structure 316, and a dial mechanism 320. The housing 311 having the components of the length adjusting element 310 is attached at a second segment end 116 of the strap element 108. The length adjusting element 310 is mounted proximate to the shoulder blades of the user, in a manner similar to spindle 202 shown by FIG. 3a.

The length adjusting element 310 provides a tension force via coil spring 323 that is fixed to axis 313. Coil spring 323 constantly biases the strap element 108 to shorten unless the dial mechanism 320 is in a locked position. In other words, the coil spring 323 will shorten the strap element 108 until the forces opposing the shorting are equal to the force exerted by the coil spring. In sum, the user of the device can wear the device, adjust their posture until they are in a correct posture while the dial mechanism 320 is unlocked, then lock the dial mechanism 320 to prevent the strap element 108 from lengthening or shortening, thereby correcting the posture of the user (e.g., for an exercise).

Dial mechanism 320 includes a dial portion 321, a handle 322, and a length adjusting element wire 324. Dial mechanism 320 may be placed anywhere on the apparatus 100 where it is readily manipulated by the user using their hand(s). The user can adjust the length of the strap element 108 of the apparatus 100 by manipulating the dial mechanism 320; rotating the handle 322 in a first direction locks the dial mechanism 320 and rotating the handle 322 of the dial mechanism 320 in a second direction 216 unlocks the dial mechanism 320. An example of a suitable location might be on the chest of the user, similar to the tightening device 140 shown in FIG. 1c.

As shown, the mounting structure 316 is attached to a housing 311 of the length adjusting element 310. A first end of the length adjusting element wire 324 extends through the spring 318 and the mounting structure 316 and is terminated by a first cap 326. A second end of the length adjusting element wire 324 extends through the dial portion 321 and is terminated by a second cap (not illustrated). In a locked position of the dial mechanism, the first cap 326 prevents gear 312 from rotating, thereby fixing the length of the strap element 108. Moreover, locking the dial mechanism may increase the tension of the strap element 108.

Referring now to FIG. 5, a perspective view of length adjusting mechanism 110 is depicted, which is configured to include a ratcheting handle 502, a release handle 504, a retracting spindle 506, and an axle 508. In one embodiment, length adjusting mechanism 110 may be disposed in any applicable location on apparatus 100 and is configured to allow user 102 to increase and decrease the length of strap element 108 and determine the preferred length that allows middle segment 114 to span the back in a snug and comfortable manner. Ratcheting handle 502 and release handle 504 may function as triggers configured to operate forwards (opening up) and backwards (closing down), as illustrated by line A. In one embodiment, ratcheting handle 502 and release handle 504 may be configured to perform a cranking motion configured to incrementally decrease slack associated with strap element 108 while interacting with retracting spindle 506. Once ratcheting handle 502 and release handle 504 open up, axle 508 is exposed, which is configured to be continuously fed strap element 108, resulting in strap element 108 interacting with retracting spindle 506. Any surplus of strap element 108 is pulled through retracting spindle 506 until tight and ratcheting handle 502 and release handle 504 are configured to cooperate to wind strap element 108 around the spindle and tighten strap element 108. In one embodiment, operation of dial 206 may be associated with the forward and backward movement of ratcheting handle 502 and release handle 504 allowing user 102 to switch from unlocked position where strap element 108 may move smoothly along retracting spindle 506 to locked position where the interaction with dial 206 engages ratcheting handle 502 and release handle 504 to close down resulting in strap element 108 being locked in place on retracting spindle 506 so that the length of strap element 108 may no longer be adjusted.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. An apparatus for enabling correct posture of a user, comprising:
(i) a first wearable harness, wherein, when worn by the user, the first wearable harness is proximate to a hip section of the user;
(ii) a second wearable harness, wherein, when worn by the user, the second wearable harness is proximate to shoulder blades of the user;
(iii) a strap element connecting the first wearable harness to the second wearable harness;
(iv) at least one length adjusting element, wherein the at least one length adjusting element adjusts a length of the strap element defined by a distance between the first wearable harness and the second wearable harness; and
wherein the at least one length adjusting element is affixed to at least one of the first wearable harness and the second wearable harness.

2. The apparatus of claim 1, wherein the second wearable harness comprises a first set of straps and a second set of straps, wherein when worn by the user :
(i) the first set of straps extend from a first straps first end proximate the shoulder blades of the user, over trapezius muscles of the user, and to a first straps second end proximate a chest of the user; and
(ii) the second set of straps extend from a second straps first end proximate the shoulder blades of the user, over a ribcage of the user, and to a second straps second end proximate the chest of the user.

3. The apparatus of claim 2, wherein the second wearable harness has the at least one length adjusting element mounted thereon and comprises a tightening device, and wherein when worn by the user, the tightening device is proximate the chest of the user.

4. The apparatus of claim 3, wherein the tightening device comprises at least one wire, the at least one wire being embedded in each of the first set of straps of the second wearable harness, and a rotating spindle engaged with the at least one wire that when rotated in a first direction tightens the at least one wire, thereby shortening the length of the strap element.

5. The apparatus of claim 3, wherein the tightening device comprises:
a rotating gear having a plurality of teeth, wherein the rotating gear rotates in a first direction and a second direction;
a drum attached to the rotating gear;
at least one wire embedded in each of the first set of straps of the second wearable harness, wherein the at least one wire is attached to the drum;
wherein the at least one wire wraps on to the drum when the drum rotates with the rotating gear in the first direction thereby tightening the at least one wire and shortening the length of the first set of straps;
wherein the at least one wire unwraps from the drum when the drum rotates with the rotating gear in the second direction thereby loosening the at least one wire and increasing the length of the first set of straps;
a pawl that engages the plurality of teeth due to a first force provided by a spring element; and
a lever connected to the pawl that disengages the pawl from the plurality of teeth when a second force acts on the lever that is greater than the first force.

6. The apparatus of claim 1, further comprising a locked position and an unlocked position, wherein in the locked position the length of the strap element is fixed, and wherein in the unlocked position the length of the strap element adjusts according to the distance between the first wearable harness and the second wearable harness.

7. The apparatus of claim 6, wherein in the locked position, the strap element provides a tension force between the first wearable harness and the second wearable harness, thereby forcing the user to maintain a correct alignment of a spine of the user when performing lifting exercises that require bending at knees of the user.

8. The apparatus of claim 6, wherein the apparatus is configured to switch between the locked position and the unlocked position by the user interacting with at least one of a dial, lever, button, or actuator.

9. The apparatus of claim 6, wherein in the unlocked position, the strap element is configured to move freely when worn by the user.

10. The apparatus of claim 1, wherein the strap element comprises a first end segment configured to be attached to the first wearable harness, a second end segment configured to be attached to the second wearable harness, and a middle segment configured to span the user's back.

11. The apparatus of claim 1, wherein the at least one length adjusting element is at least one of a spindle, spool, and racketing system configured to increase and decrease the length of the strap element between the first wearable harness and the second wearable harness.

12. The apparatus of claim 11, wherein the at least one length adjusting element comprises:
(a) a gear having a plurality of teeth ;
(b) a mounting structure attached to a housing of the at least one length adjusting element
(c) a spring attached to the mounting structure;
(d) a dial mechanism comprising a dial portion a handle, and a length adjusting element wire, wherein:
(i) a first end of the length adjusting element wire extends through the spring and the mounting structure, and is terminated by a first cap;
(ii) a second end of the length adjusting element wire extends through the dial portion and is terminated by a second cap (not illustrated); and
(iii) rotating the handle in a first direction locks the dial mechanism and rotating the handle of the dial mechanism in a second direction unlocks the dial mechanism.

13. The apparatus of claim 1, wherein each of the first wearable harness and the second wearable harness comprises at least one strap configured to attach the first wearable harness and the second wearable harness to the user's body.

14. The apparatus of claim 1, the length of the strap element between the first wearable harness and the second wearable harness is adjustable to properly fit the user.

15. An apparatus for enabling correct posture of a user, comprising:
(i) a first wearable harness, wherein, when worn by the user, the first wearable harness is proximate to a hip section of the user;
(ii) a second wearable harness, wherein, when worn by the user, the second wearable harness is proximate to shoulder blades of the user;
(iii) a strap element connecting the first wearable harness to the second wearable harness;
(iv) at least one length adjusting element, wherein the at least one length adjusting element adjusts a length of the strap element defined by a distance between the first wearable harness and the second wearable harness;
(v) wherein the second wearable harness comprises a first set of straps and a second set of straps, wherein when worn by the user (a) the first set of straps extend from a first straps first end proximate the shoulder blades of the user, over trapezius muscles of the user, and to a first straps second end proximate a chest of the user, and (b) the second set of straps extend from a second straps first end proximate the shoulder blades of the user, over a ribcage of the user, and to a second straps second end proximate the chest of the user;
(vi) wherein the second wearable harness comprises a tightening device that comprises:
(a) a rotating gear having a plurality of teeth, wherein the rotating gear rotates in a first direction and a second direction;
(b) a drum attached to the rotating gear;
(c) at least one wire embedded in each of the first set of straps of the second wearable harness, wherein the at least one wire is attached to the drum;
(I) wherein the at least one wire wraps on to the drum when the drum rotates with the rotating gear in the first direction thereby tightening the at least one wire and shortening the length of the first set of straps;
(II) wherein the at least one wire unwraps from the drum when the drum rotates with the rotating gear in the second direction thereby loosening the at least one wire and increasing the length of the first set of straps;
(d) a pawl that engages the plurality of teeth due to a first force provided by a spring element; and
(e) a lever connected to the pawl that disengages the pawl from the plurality of teeth when a second force acts on the lever that is greater than the first force.

16. The apparatus of claim 15, further comprising a locked position and an unlocked position, wherein in the locked position the length of the strap element is fixed, and wherein in the unlocked position the length of the strap element adjusts according to the distance between the first wearable harness and the second wearable harness.

17. The apparatus of claim 16, wherein in the locked position, the strap element provides a tension force between the first wearable harness and the second wearable harness, thereby forcing the user to maintain a correct alignment of a spine of the user when performing lifting exercises that require bending at knees of the user.

18. The apparatus of claim 16, wherein the apparatus is configured to switch between the locked position and the unlocked position by the user interacting with at least one of a dial, the lever, a button, or an actuator.

19. The apparatus of claim 16, wherein in the unlocked position, the strap element is configured to move freely when worn by the user.

20. A method for maintaining a correct posture of a user while performing an exercise comprising:
mounting a first wearable harness on a hip section of the user;
mounting a second wearable harness on an upper body of the user;
positioning, longitudinally along a back portion of the user, a strap element that connects the first wearable harness to the second wearable harness, wherein the strap element defines a length defined by a distance between the first wearable harness and the second wearable harness; and
adjusting the length between the first wearable harness and the second wearable harness so that the strap element forces the user to maintain the correct posture while performing the exercise.
